(19) United States
(12) Patent Application Publication
(10) Pub. No.: US 2005/0009030 A1
(43) Pub. Date: Jan. 13, 2005

(54) Histone deacetylase: novel molecular target of neurotoxicity

(76) Inventors: Fabien Schweighoffer, Vincennes (FR); Annelies Resink, Na Laren Nh (NL)

Correspondence Address: NIXON & VANDERHYE, PC, 1100 N GLEBE ROAD, 8TH FLOOR, ARLINGTON, VA, 22201-4714, US

(21) Appl. No.: 10/502,754

(22) PCT Filed: Mar. 25, 2003
(86) PCT No.: PCT/FR03/00940

Related U.S. Application Data
(60) Provisional application No. 60/372,116, filed on Apr. 15, 2002.

(30) Foreign Application Priority Data
Mar. 26, 2002 (FR) 02 03792

(51) Int. Cl. C12Q 1/68

(52) U.S. Cl. 435/6

(57) ABSTRACT

The present invention concerns the field of biology, genetics and medicine. It particularly pertains to new methods for detecting, characterising and/or treating neurodegenerative diseases, particularly amyotrophic lateral sclerosis. The invention also pertains to methods for identifying or screening for compounds active in these diseases. The invention also relates to the compounds, genes, cells, plasmids or compositions useful for implementing said methods. The invention particularly describes the role of the histone deacetylases, and particularly histone deacetylase 2, in these diseases and its use as a therapeutic, diagnostic or experimental target.

The present invention pertains to the field of biology, genetics and medicine. More particularly, it relates to novel methods for detecting, characterising and/or treating neurodegenerative diseases, particularly amyotrophic lateral sclerosis. The invention also relates to methods for identifying or screening compounds that are active in these diseases. The invention also relates to the compounds, genes, cells, plasmids or compositions that can be used to carry out the above methods. The invention is particularly premised on the identification of the role, in these diseases, of enzymes involved in the phosphorylation of nuclear factors, of which the histones should particularly be mentioned, and describes their use as therapeutic, diagnostic or experimental targets or markers of these disorders.

Many neurodegenerative diseases have been described as having a component or stage associated with excitotoxicity. This is the case for Alzheimer's disease, Parkinson's disease, multiple sclerosis and Huntington's chorea.

Amyotrophic lateral sclerosis (ALS) is a neurodegenerative disease associated with various types of inclusion bodies such as Lewy bodies and characterised by apoptosis of the spinal and cortical motoneurons, the fatal outcome of which is occasionally associated with frontal dementia. There are sporadic forms of the disease, with no described mutation, and familial forms (FALS) associated with mutations of the SOD1 gene that codes for superoxide dismutase. The majority of cases are sporadic, the familial forms (FALS) being very rare. It is likely that a long asymptomatic period precedes the onset of clinical symptoms. The latter are variable and their classification is complex. Future therapeutic developments will replace treatment of the symptoms with strategies based on the molecular causes of the disease. At the cellular level, these symptoms are associated with death of the cortical motoneurons and the spinal motoneurons. This neuronal cell death has been linked with various phenomena that form the basis of several neurodegenerative diseases. This is so for glutamate-related excitotoxicity, oxidative stress, a certain type of autoimmune disease directed against neuronal markers (calcium channels in the case of ALS) as well as abnormalities of the cytoskeleton. Even though these phenomena have been described, the cause or causes of these diseases, including ALS, remain obscure. Furthermore, even though FALS are associated with mutations in the SOD1 gene that encodes superoxide dismutase, the mechanisms that commit the neurons to cell death, at least one component of which is apoptosis, are unknown.

The identification of the molecular events involved in the different phenomena that are involved in cell death will enable the design of novel therapeutic strategies. It is not easy to study these events using human biopsy material. These biopsy materials obviously come from post-mortem samples, the quality of which is difficult to verify, and they only represent pathological states that correspond to the later stages of the disease.

Animal models provide access to biological samples that enable different stages of development of a disease to be analysed and compared to healthy controls. In this regard, transgenic mice expressing the human SOD1 gene carrying one of the mutations that is prevalent in FALS (mutation G93A) are available from Jackson Laboratory, on condition that a license for uses thereof is obtained from NorthWestem University. Within 120 days, this model reproduces the fatal outcome of the disease with symptoms comparable to those of the human disease. The onset of the symptoms of ALS associated with the SOD1 G93A mutation is not the consequence of a reduction in superoxide dismutase activity, but a gain in function, which increases the capacity of the enzyme to generate free radicals. In spite of these observations, the molecular events that are responsible for the different stages of ALS are poorly understood. The complexity of these molecular events reflects the progression of the disease: in the transgenic model studied, no neuronal deregulation or clinical manifestation has been reported by day 30. Day 60 corresponds to a stage that just precedes the first symptoms, but that is already characterised at the cerebral level by changes in cell physiology, such as alteration of mitochrondrial metabolism, stress and neuronal cell death associated with excitotoxicity. At 90 days, 50% of the cortical and spinal motoneurons are dead and an active process of neuronal apoptosis begins, in parallel with astrocyte activation. Excitotoxicity is no longer seen at this stage. Neuronal cell death is associated here with the activation of caspases, which do not appear to be involved in the early stages of the disease.

Besides this animal model, it is also possible to study excitotoxicity using cell-based models. Cerebellar granule cells represent a valuable model for studying experimental neuronal cell death. Indeed, cultures of these neurons exhibit an enrichment of neurons with respect to glial cells, which enables the events that are specific to neuronal cell death to be studied preferentially.

Several approaches can be envisioned in order to reproduce certain aspects of excitotoxicity. The neurons can be treated with glutamate, which will stimulate all the receptors for this excitatory amino acid, the metabotropic receptors and the ionotropic receptors. Treatments using NMDA or kainate can also be applied in order to stimulate their respective ionotropic receptors. One of the consequences of stimulating these ionotropic receptors is an efflux of potassium when these channels open. This efflux of potassium directly contributes to excitotoxicity, because cell death is reduced by increasing the extracellular concentration of potassium.

Reciprocally, culturing neurons in the presence of low concentrations of potassium causes them to die.

Consequently, one of the components of excitotoxicity can be studied by culturing neurons in the presence of low concentrations of potassium.

The identification of the various molecular events that are specific to this phenomenon should enable new therapeutic targets to be identified, as well as new diagnostic markers.

The present invention now describes the identification of genetic events involved in excitotoxicity and neuronal cell death phenomena. The present invention thus provides new therapeutic and diagnostic approaches for diseases associated with these phenomena, as well as new targets for the identification of active compounds.

More particularly, a qualitative differential analysis was performed using RNA extracted from rat cerebellar granule cells cultured according to techniques known to those skilled in the art. Advantageously, RNA extracts from viable neurons cultured in the presence of 25 μM potassium were compared to RNA extracts from neurons grown under toxic conditions, by lowering the potassium concentration to 5 μM. This analysis was performed using qualitative differential screening, using the DATAS technique (described in application no WO99/46403).

This analysis allowed the identification of a cDNA fragment derived from the mRNA of a histone deacetylase, demonstrating the involvement of this enzyme in the development of excitotoxicity and neuronal cell death processes. More particularly, the present application demonstrates the existence of a modification of the splicing of histone deacetylase during the course of excitotoxicity phenomena. The results obtained demonstrate an alteration of the coding sequence of this enzyme in brains that were engaged in excitotoxicity at the time of the onset of the symptoms of ALS. This alteration suggests an increase in the activity of the corresponding enzyme and therefore hypoacetylation of histones and other nuclear targets.

The level of acetylation of these targets regulates transcriptional activity and splicing of pre-mRNA. The level of acetylation of histones and other nuclear factors is regulated by the balance between two enzymatic activities: histone acetyltransferase activity and histone deacetylase activity.

It has been shown in the case of diseases associated with aggregation of proteins that have been modified by the addition of glutamine repeats (huntingtin in Huntington's chorea, the androgen receptor in the case of certain types of spinal or bulbar muscular atrophy) that histone acetyltransferases are sequestered in the protein aggregates. Application no WO01/17514 proposes complex compositions comprising a gene expression inducing agent and a modulator of histone acetylation, to control the expression of genes regulated by phosphorylation. These compositions are based on a non-specific action, at the level of chromatin, and do not suggest any implication of histone deacetylases in the mechanisms of neurotoxicity, nor any strategy based on the regulation of these enzymes for treating such pathological conditions. Application WO00/71703 relates to the use of antisense oligonucleotides specific for histone deacetylases for treating cancers.

Surprisingly, the invention demonstrates, for the first time, the existence of a molecular mechanism of genetic deregulation that leads to excitotoxicity by decreasing the acetylation of histones and other nuclear factors involved in the regulation of gene expression.

The present invention thus provides a new molecular target for the development of therapeutic and diagnostic approaches for diseases associated with excitotoxicity. These strategies are based on modulating histone acetylation activity, and are more particularly based on increasing the level of histone acetylation. This modulation can be effected in a variety of ways, preferably by the use of histone deacetylase inhibitors. These therapeutic strategies enable neuronal viability to be improved during the excitotoxic phenomena involved in various diseases of the nervous system, particularly Alzheimer's disease, multiple sclerosis and amyotrophic lateral sclerosis (ALS).

It is an object of the invention to use a histone deacetylase inhibitor for preparing a composition for treating neurodegenerative diseases, particularly during the early stages, more preferably for reducing neuronal excitotoxicity associated with neurodegenerative diseases, particularly during the early stages.

It is another object of the invention to provide a method for treating a disease associated with neuronal stress, particularly excitotoxicity, comprising administering a histone deacetylase inhibitor to a subject.

As used in the invention, disease associated with excitotoxicity more preferably means a neurodegenerative disease chosen from among ALS, Alzheimer's disease, Parkinson's disease, multiple sclerosis and cerebral ischaemia. The invention is also applicable to the treatment of neuronal excitotoxicity arising in other diseases, particularly during the early stages thereof.

In the context of the invention, the term “treatment” denotes preventive, curative or palliative treatment, as well as patient management (reducing suffering, improving life expectancy, slowing the progression of the disease, improving the viability of neurons under conditions of excitotoxicity, etc.). The treatment can moreover be carried out in combination with other agents or treatments, particularly those that address late disease events, such as caspase inhibitors or other active compounds.

The term “inhibitor” denotes any compound or treatment capable of inhibiting or reducing the expression or activity of a histone deacetylase. The inhibitor is preferably selective, i.e., its activity is essentially directed against a histone deacetylase, with no substantial direct activity on any other enzyme. In particular, it is essential that the histone deacetylase inhibitor should have no direct inhibitory effect on a histone acetyltransferase.

Various histone deacetylases have been identified, cloned and characterised. The following can be particularly cited: histone deacetylase-1 (HDAC1), histone deacetylase-2 (HDAC2) and histone deacetylase-3 (HDAC3), particularly the human enzymes. The nucleic acid sequences encoding these enzymes and the corresponding amino acids sequences are described in the prior art, for example in the GenBank data banks under the references NM_{—}004964 (hHDAC1); NM_{—}001527 (hHDAC2); NM_{—}003883 (hHDAC3); AF006603 (mHDAc2). These sequences are also available from other public sources. It should be understood that the invention also pertains to natural variants and/or homologues of these specific sequences.

Within the scope of the present invention, an inhibitor of a human histone deacetylase is preferentially used, particularly an inhibitor of hHDCA1, hHDCA2 and/or hHDCA3, more preferably an inhibitor compound.

The compound used can be any compound capable of inhibiting the expression of the histone deacetylase, i.e., in particular any compound that inhibits transcription of the gene, RNA maturation, RNA translation, post-translational modification of the protein, the binding of the protein to a molecular target, etc.

The compound can be of varied nature and origin, such as, particularly of natural origin (for example of plant, bacterial, viral, animal or eukaryotic origin) or synthetic (particularly an organic or inorganic, synthetic or semi-synthetic molecule). It can for example be a nucleic acid, a polypeptide (or a protein or peptide), a lipid or a chemical compound, etc.

In a first embodiment, the inhibitor is an antisense nucleic acid capable of inhibiting transcription of the histone deacetylase gene or translation of the corresponding messenger. The antisense nucleic acid can comprise all or part of the sequence of the histone deacetylase gene, the histone deacetylase messenger, or of a sequence that is complementary thereto. The antisense sequence can be a DNA, an RNA, a ribozyme, etc. It may be single-stranded or double-stranded. It can also be a RNA encoded by an antisense gene. When an antisense nucleic acid comprising part of the sequence of the gene or messenger under consideration is being used, it is preferred to use a part comprising at least 10 consecutive bases from the sequence, more preferably at least 15, in order to ensure specific hybridisation. In the case of an antisense oligonucleotide, it typically comprises less than 100 bases, for example in the order of 10 to 50 bases. This oligonucleotide can be modified to improve its stability, its nuclease resistance, its cell penetration, etc. Perfect complementarity between the sequence of the antisense molecule and that of the target gene or messenger is not required, but is generally preferred.

According to another embodiment, the inhibitor compound is a polypeptide. It may be, for example, a peptide comprising a region of a histone deacetylase sequence, and capable to antagonise its activity. A peptide advantageously comprises from 5 to 50 consecutive amino acids of the primary sequence of the deacetylase under consideration, typically from 7 to 40. The polypeptide can also be an anti-histone deacetylase antibody, or a fragment or derivative of such an antibody, for example a Fab fragment, a CDR region, or, more preferably, a single chain antibody (e.g. ScFv). Single chain antibodies are particularly advantageous insofar as they can act in a specific and intracellular fashion to modulate the activity of a target protein. Such antibodies, fragments, or derivatives can be produced by conventional techniques comprising immunising an animal and recovering the serum (polyclonal) or spleen cells (in order to produce hybridomas by fusion with appropriate cell lines).

Methods for producing polyclonal antibodies in various species are described in the prior art. Typically, the antigen is combined with an adjuvant (e.g. Freund's adjuvant) and administered to an animal, typically by subcutaneous injection. Repeated injections can be performed. Blood samples are collected and the immunoglobulin or serum is separated. Conventional methods for producing monoclonal antibodies comprise immunising of an animal with an antigen, followed by recovery of spleen cells, which are then fused with immortalised cells, such as myeloma cells. The resulting hybridomas produce monoclonal antibodies and can be selected by limiting dilution in order to isolate individual clones. Fab or F(ab′)2 fragments can be produced by protease digestion, according to conventional techniques.

According to another embodiment, the compound is a chemical compound, of natural or synthetic origin, particularly an organic or inorganic molecule, capable of modulating the expression or the activity of a histone deacetylase. Such compounds can be produced and/or selected according to various assays described below. By way of a preferred non-limiting example, trichostatin A, trapoxin, apicidin, pyroxamide, valproate, benzamide, various butyrates, such as sodium butyrate or phenylbutyrate, butyrate derivatives as described in application WO98/00127, as well as analogues and the like may be cited. Trichostatin A is a preferred example.

It is a particular object of the invention to use a human histone deacetylase inhibitor compound, particularly a selective inhibitor, for preparing a composition for use in reducing neuronal excitotoxicity.

It is a particular object of the invention to use a human histone deacetylase 2 inhibitor compound, particularly a selective inhibitor, for preparing a composition for use in reducing neuronal excitotoxicity, particularly for the treatment of ALS.

It is a particular object of the invention to use a human histone deacetylase inhibitor, particularly a selective inhibitor, for preparing a composition for treating ALS, particularly for reducing neuronal excitotoxicity during the early stages of ALS.

It is another particular object of the invention to use trichostatin A for preparing a composition for use in reducing neuronal excitotoxicity and/or for preparing a composition for treating ALS.

It is another particular object of the invention to use trichostatin A for preparing a composition for use in inhibiting the activity of histone deacetylase 2 in patients with ALS.

The invention also relates to methods for treating ALS comprising administering to a subject an effective amount of a compound that inhibits the expression or activity of a histone deacetylase, particularly histone deacetylase 2, preferably human.

Preferably, the invention is used for treatment during the early stages of neurodegenerative diseases.

Administration can be performed by any method known to those skilled in the art, preferably by injection, typically by intraperitoneal, intracerebral, intravenous, intra-arterial or intramuscular route. These injected doses can be adapted by one skilled in the art. Typically for inhibitor compounds of a chemical nature, between about 0.01 mg and 100 mg/kg are injected. For nucleic acid compounds, the doses can vary for example between 0.01 mg and 100 mg per dose. It is understood that repeated injections may be performed, possibly in combination with other active agents or any pharmaceutically acceptable carrier (e.g., buffers, saline or isotonic solutions, in the presence of stabilising agents, etc.).

The invention can be used in mammals, particularly in humans.

The invention particularly shows the existence of splicing events affecting the coding region of histone deacetylase 2 (mHDA2, GenBank reference: AF006603), more particularly a region spanning nucleotides 2934 to 3243. This splicing event, which is preferentially detected under conditions of neuronal viability (25 μM potassium) inactivates the enzyme and results in increased acetylation of histones and other factors involved in gene expression in the viable neurons. Reciprocally, it has been demonstrated that a reduction in acetylation of the same nuclear factors is associated with neuronal cell death in the presence of 5 μM potassium. The present invention therefore opens up the possibility of a new therapeutic strategy based on the use of histone deacetylase inhibitors in order to restore neuronal viability during potassium efflux and particularly during excitotoxic phenomena and more particularly in diseases such as ALS. The present invention proposes, for the first time, histone deacetylase 2 as a therapeutic target for treating the molecular events associated with excitotoxicity. According to particular embodiments, the invention can be used for inhibiting or reducing neuronal excitotoxicity during the early stages of neurodegenerative diseases. It is particularly applicable to the treatment of Alzheimer's disease, Parkinson's disease, multiple sclerosis, or cerebral ischaemia.

The present invention also provides a new target for identifying, validating, selecting or optimising active compounds. The invention indeed enables the selection of compounds with advantageous therapeutic or biological properties, on the basis of their capacity to modulate the expression or activity of histone deacetylase. These assays can be performed in a cell-based or animal system, or a cell-free system (e.g., using isolated proteins, polypeptides or nucleic acids, or on in vitro expression systems), and can be based on the measure of an interaction (e.g., binding, displacement or competition assays, etc.) or of a function (activity, transcription, etc.).

Another particular object of the invention relates to a method for selecting, identifying or characterising active compounds, particularly in diseases associated with excitotoxicity or neuronal stress, comprising contacting a test compound with a cell expressing a histone deacetylase or a fragment or functional variant thereof, and determining the capacity of the compound to inhibit the expression or activity of this protein.

The methods can be carried out using different cell populations, such as primary cells or cell lines of mammalian origin (human, murine, etc.). Advantageously cells are used that do not naturally express the histone deacetylase in question, but that are transfected with a nucleic acid encoding the desired enzyme. In this way, the selectivity of the method is increased. Lower eukaryotic cells (yeast, etc.) or prokaryotic cells can also be used. Non-human transgenic animals may also be used.

The inhibitory effect can be measured in various ways, such as, particularly by assaying the RNA, assaying the protein, measuring an activity, etc. These can be measured using any conventional immuno-enzymatic technique (RIA, ELISA, EIA, etc.) or by hybridisation techniques using labelled probes or chips, or by amplification using specific primers, etc.

Selection methods can also be performed in a cell-free system, by measuring the capacity of test compounds to bind a histone deacetylase or a variant or fragment thereof. In connection with this, another particular object of the invention relates to a method for selecting, identifying or characterising active compounds, particularly against diseases associated with excitotoxicity or neuronal stress, comprising contacting a test compound with a histone deacetylase or a variant or fragment thereof, and determining the capacity of the test compound to bind to said histone deacetylase, fragment or variant. The histone deacetylase, fragment or variant can be used in an isolated and purified form, a soluble form, or attached to a matrix (e.g. a bead or column, etc.), or incorporated into a membrane or vesicle. Binding of the test compound to the histone can be determined by any known technique, particularly by displacement of a labelled reference ligand, by gel migration or electrophoresis, etc.

The term “variant” includes polypeptides comprising one or several mutations, substitutions, deletions and/or additions of one or several amino acid residues and that have substantially the same antigenic specificity or activity, and that particularly retain the capacity to deacetylate a histone. Typical examples of derivatives include sequence variations due to histone deacetylase polymorphism, to splicing, etc. Particularly preferred derivatives contain at most 5 amino acid residues that differ from those present in the wild-type sequence. The term “fragment” denotes any polypeptide comprising from 5 to 100 consecutive residues from the histone deacetylase amino acid sequence, preferably from 10 to 100. The fragments advantageously include an active site of the enzyme.

Test compounds can be of diverse nature and origin, such as natural or synthetic compounds, lipids, nucleic acids, polypeptides, chemicals, etc. They can be isolated compounds or in a mixed form, combinatorial libraries, etc. In the methods of the invention, it is possible to test several test compounds in parallel, for example in suitable devices such as multi-well plates, boxes, etc.

Another object of the invention relates to any splice variant of a histone deacetylase as well as any nucleic acid encoding such a polypeptide, vectors containing the same, recombinant cells, and their use. Vectors can be plasmids, phages, cosmids, viruses, artificial chromosomes, etc. Preferred vectors are for example plasmid vectors, such as those derived from commercial plasmids (pUC, pcDNA, pBR, etc.). Such vectors advantageously contain a selection gene and/or an origin of replication and/or a transcriptional promoter. Other particular vectors are, for example, viruses or phages, particularly recombinant viruses that are replication-defective, such as viruses derived from retroviruses, adenovirus, MV, herpes virus, baculovirus, etc. The vectors can be used in any competent host, such as, for example prokaryotic or eukaryotic cells. These may be bacteria (for example E. coli), yeasts (for example Saccharomyces or Kluyveromyces), plant cells, insect cells, mammalian cells, particularly human cells, etc. They can be cell lines, primary cells, mixed cultures, etc

The present invention can also be used for diagnosing situations of excitotoxicity, particularly in early stages. It is particularly useful for detecting the presence, predisposition to or development of an excitotoxic situation in a subject, particularly a disease associated with such a situation, such as Alzheimer's disease, Parkinson's disease, multiple sclerosis, ALS or cerebral ischaemia. It is particularly adapted to detecting multiple sclerosis or ALS at an early stage.

Another aspect of the present application therefore relates to methods and tools for detecting the presence, in biological samples, of a histone deacetylase (or a splice variant or other genetic alteration thereof), or for dosing or determining its relative quantities.

A particular object of the invention relates to a method for detecting a situation of excitotoxicity or neuronal stress in a subject, comprising measuring in vitro or ex vivo the expression of one (or several) histone deacetylases, particularly histone deacetylase 2, in a sample taken from the subject.

Another particular object relates to a method for detecting a situation of excitotoxicity or neuronal stress in a subject, comprising detecting the presence (or absence) of a mutated form of one (or several) histone deacetylases, particularly histone deacetylase 2, or the corresponding RNA, in a sample taken from the subject.

Suitable tools for measuring or detecting a protein, RNA or expression particularly include probes or nucleic acid primers, antibodies or other specific ligands, kits, devices, chips, etc. The detection methods can include hybridisation, PCR, chromatographic or immunological methods, etc. These methods are particularly suitable for detecting, characterising, monitoring the progression of or the efficacy of a treatment in diseases such as those mentioned herein above, or for determining a predisposition to a disease.

The method can be carried out using various biological samples, such as blood, plasma, urine, serum, saliva, biopsy material or cell cultures, etc. It can preferably be a sample containing nerve or muscle cells. Depending on the technique used, the sample can undergo a prior treatment, for example to render the nucleic acids available to a hybridisation and/or an amplification reaction, and/or to render the proteins available to an immunological or enzyme reaction.

In a particular embodiment, the expression in the sample of mRNA encoding a histone deacetylase is measured, or the presence or quantity of the mRNA is detected or assayed. Several histone deacetylases can be detected or assayed in parallel.

This measurement, detection or assay can be performed by hybridisation of the sample with a nucleic acid probe specific for the RNA under consideration, particularly a probe comprising all or part of a sequence from the messenger RNA of histone deacetylase or a complementary or derived sequence. In a particular embodiment, the probe is single-stranded and/or is labelled, in order to facilitate the detection of the hybridisation product. The labelling can be radioactive, fluorescent, luminescent, etc. The probe can be immobilised on a matrix.

It is a particular object of the invention to use a nucleic acid comprising all or part of a sequence derived from the gene or messenger RNA of a histone deacetylase to carry out a method for diagnosing, detecting, screening for or characterising a situation of neuronal stress and more particularly an excitotoxic situation, particularly a method for diagnosing, detecting, screening for or characterising neurodegenerative diseases with a component or stage associated with excitotoxicity or neuronal stress, notably a sequence that is specific for, complementary to or derived from this sequence.

As indicated herein above, the present application demonstrates the existence of an unspliced form in certain tissues engaged in neurotoxic processes, and the invention enables the detection or relative dosage of the spliced form and the unspliced form in the sample. The onset, presence or increased level of the unspliced species is correlated with the development of the excitotoxic situation. In a particular variant, the method of the invention therefore includes an analysis of the presence of the spliced form and/or the unspliced form of the histone deacetylase RNA. This detection can be performed for example by using a nucleic acid probe specific for the sequence resulting from the junction between the non-deleted (i.e. unspliced) regions of the RNA. The change in the ratio between the spliced form and the unspliced form can be monitored as an indicator of the progression of the disease (or the efficacy of a treatment).

The measurement, detection or assay can also be performed by selective amplification of the nucleic acids in the sample using a nucleic acid primer (or a primer pair) specific for the RNA under consideration. In a particular embodiment, the primer (or one of the primers of the pair) is specific for the sequence resulting from the junction between the non-deleted (i.e. unspliced) regions of the RNA.

Amplification can be performed, for example using PCR. The amplification product can be detected or assayed by any known technique. Such a primer (or primer pair) constitutes another object of the present application.

In another particular embodiment, the presence or quantity of histone deacetylase in the sample is detected or assayed. This detection can be performed using a specific antibody, or any other specific ligand.

Another object of the invention relates to a (product comprising a) matrix on which one or several nucleic acids (including a vector, probe, primer, oligonucleotide, antisense sequence), polypeptides (including antibody) or cells as defined herein above are immobilised. The matrix can be solid, flat or not, uniform or not, such as for example nylon, glass, plastic, etc., or any other compatible material. The polypeptides or nucleic acids are preferably immobilised by one end, under conditions that leave the molecule accessible for a reaction involving interaction with a specific ligand, such as an antibody or a probe. The polypeptides or nucleic acids can be arranged in a precise manner on the matrix, and deposited several times over.

The invention can also be used for characterising tissue and ischaemic situations. This use is also based on detecting or assaying a histone deacetylase or an altered form in the tissue.

Other aspects and advantages of the present invention will be apparent on reading the following examples, which should be considered as illustrative and non-limiting.

### EXAMPLES

### Example 1

### Identification of Histone Deacetylase 2 as a Molecular Target for Excitotoxicity

Qualitative differential analysis was performed using polyadenylated (poly A+) RNA extracts from samples of animal brains corresponding to different stages of the disease. The neurons were not previously isolated, in order to take into account a maximum number of alternative splicing events associated with the development of the disease.

The poly A+ RNA is prepared using techniques known to those skilled in the art. In particular, it can involve treatment with chaotropic agents such as guanidinium thiocyanate followed by extraction of the total RNA by means of solvents (phenol or chloroform, for example). Such methods are well known to those skilled in the art (see Maniatis et al., Chomczynsli et al., Anal. Biochem. 162 (1987)156), and can be carried out easily using commercially available kits.

Poly A+ RNA is prepared from this total RNA according to conventional methods known to those skilled in the art and available in commercial kit form.

This poly A+ RNA is used as a template for reverse transcription reactions using reverse transcriptase. Advantageously, the reverse transcriptases used have no RNase H activity, which results in the initial strands of complementary DNA obtained being longer than those obtained with conventional reverse transcriptases. Such reverse transcriptase preparations with no RNase H activity are commercially available.

For each time point for the kinetics of disease development (30 days, 60 days and 90 days), the poly A+ RNA and single-stranded cDNA are prepared from transgenic animals (T) and syngeneic control animals (C).

In accordance with the DATAS technique, hybridisations are performed for each time point of the kinetics between mRNA (C) and cDNA (T), as are reciprocal hybridisations between mRNA (T) and cDNA (C).

These mRNA/cDNA heteroduplexes are then purified according to DATAS technique protocols.

The RNA sequences that are not paired with complementary DNA are freed from these heteroduplexes by the action of RNase H, as this enzyme degrades unpaired RNA sequences. These unpaired sequences represent the qualitative differences that exist between RNA molecules that are otherwise homologous. These qualitative differences can be localised anywhere in the sequence of the RNA molecules, either 5′, 3′ or in the middle of the sequence and particularly in the coding sequence. Depending on their localisation, these sequences can not only be modifications due to splicing, but also the consequence of translocations or deletions.

The RNA sequences that represent qualitative differences are then cloned according to techniques known to those skilled in the art and particularly those described in the DATAS technique patent.

These sequences are grouped into cDNA libraries that constitute the qualitative differential libraries. One of these libraries contains the exons and introns specific to the healthy situation; the other libraries contain the splicing events that are characteristic of the pathological conditions.

The differential expression of the clones was checked by hybridisation with probes obtained by reverse transcription of messenger RNA molecules extracted from the various situations studied. The clones that show differential hybridisation are retained for later analysis. The sequences identified by DATAS correspond to introns and/or exons that are differentially expressed in the disease situation compared to the healthy situation, in terms of their splicing. These splicing events can be specific to a given stage of development of the disease or characteristic of the healthy state.

Comparison of these sequences with the data banks enables the information obtained to be classified, and to propose a sounded selection of the sequences, according to their diagnostic or therapeutic value.

The results obtained showed the existence of splicing events that affect the coding region of histone deacetylase 2 (mHDA2, GenBank reference: AF006603), more particularly a region spanning nucleotides 2934 to 3243. This splicing event, that was preferentially detected under conditions of neuronal viability (25 μM potassium) inactivates the enzyme and results in increased acetylation of histones and other factors involved in gene expression in viable neurons. Reciprocally, it has been demonstrated that decreased acetylation of the same nuclear factors is associated with neuronal cell death in the presence of 5 μM potassium.

### Example 2

### Inhibition of Excitotoxicity by Histone Deacetylase Inhibitors

For this example, rat cerebellar granule cells were placed in culture, according to techniques known to those skilled in the art. Excitotoxicity was induced in these cells by two types of treatment: firstly, by the joint administration of 100 μM NMDA (N-Methyl-D-aspartic acid) and 10 μM serine, and secondly by administration of 50 μM kainate. Under the experimental conditions adopted, 30 to 40% toxicity was observed and measured using MTT tests, known to those skilled in the art.

Inhibition of excitotoxicity can be demonstrated in the presence of histone deacetylase inhibitors, particularly trichostatin A. The present invention therefore documents the involvement of histone deacetylase 2 in excitotoxic mechanisms, particularly in an ALS model, and also the capacity of inhibitors of this enzyme to preserve neuronal viability during stress associated with excitotoxicity.

Other aspects and applications of the invention lie in:
the use of any nucleic acid fragment, including antisense RNA, with the aim of inhibiting histone deacetylase 2 expression in patients with such diseases,
the use of any chemical compound, trichostatin A, or any pharmaceutical composition containing it, with the aim of inhibiting histone deacetylase 2 expression in patients with such diseases.

1-18. canceled.

19. A method for detecting a situation of excitotoxicity or neuronal stress in a subject, comprising measuring in vitro the expression of a histone deacetylase in a sample from the subject, wherein a deregulated expression is indicative of a situation of excitotoxicity or neuronal stress in said subject.

20. The method of claim 19, wherein measuring the expression of the histone deacetylase comprises detecting the presence of a mutated form of a histone deacetylase or of its corresponding RNA.

21. The method of claim 19, wherein measuring the expression is performed by hybridisation of the sample with a nucleic acid probe that is specific for said histone deacetylase RNA and determining the presence of hybrids.

22. The method of claim 3, wherein the nucleic acid probe comprises all or part of the sequence of a histone deacetylase messenger RNA or of a sequence that is complementary to or derived from this sequence.

23. The method of claim 19, wherein measuring the expression is performed by selective amplification of the nucleic acids in the sample using a nucleic acid primer (or a primer pair) that is specific for said RNA and determining the presence of an amplification product.

24. The method of claim 19, wherein said histone deacetylase is histone deacetylase 2.

25. The method of claim 19, wherein the sample comprises nerve or muscle cells.

26. The method of claim 19, for diagnosing or detecting Alzheimer's disease, Parkinson's disease, multiple sclerosis, ALS or cerebral ischaemia.

27. The method of claim 26, for detecting multiple sclerosis at an early stage thereof.

28. The method of claim 22, wherein the nucleic acid comprises the sequence of histone deacetylase 2 mRNA or a sequence that is complementary thereto.

29. The method of claim 22, wherein the nucleic acid comprises the sequence of a junction region resulting from splicing of a domain in a mRNA encoding histone deacetylase 2, or a sequence that is complementary thereto.

30. A method for treating a neurodegenerative disease associated with excitotoxicity, comprising administering to a subject in need thereof an effective amount of a histone deacetylase inhibitor compound.

31. The method of claim 30, wherein the compound is an antisense nucleic acid that inhibits transcription of the histone deacetylase gene or translation of the corresponding messenger RNA.

32. The method of claim 30, wherein the compound is a chemical compound, selected from trichostatin A, trapoxin, apicidin, pyroxamide, valproate, benzamide, butyrates, butyrate derivatives, and analogues thereof.

33. The method of claim 30, for inhibiting or reducing neuronal excitotoxicity during early stages of said neurodegenerative disease.

34. The method of claim 30, wherein said neurodegenerative disease is Alzheimer's disease, Parkinson's disease, multiple sclerosis or cerebral ischaemia.

35. The method of claim 33, for reducing neuronal excitotoxicity during the early stages of ALS.

36. A method for selecting, identifying or characterising compounds active against neurodegenerative diseases, comprising contacting a test compound with a cell expressing a histone deacetylase or a fragment or functional variant thereof, and determining the capacity of the compound to inhibit the expression or activity of this protein, said capacity being an indication that the compound is active against neurodegenerative diseases.

37. A method for selecting, identifying or characterising compounds active against neurodegenerative diseases, comprising contacting a test compound with a histone deacetylase or a variant or fragment thereof, and determining the capacity of the test compound to bind said histone deacetylase, fragment or variant, said binding being an indication that the compound is active against neurodegenerative diseases.
